(19) United States
(12) Patent Application Publication
(10) Pub. No.: US 2012/0088678 A1
(43) Pub. Date: Apr. 12, 2012

(54) METHOD FOR PREDICTION OF RESPONSE TO RHEUMATOID ARTHRITIS THERAPEUTICS

(75) Inventor: Salvatore Albani, La Jolla, CA (US)

(73) Assignee: Sanford-Burnham Medical Research Institute, La Jolla, CA (US)

(21) Appl. No.: 13/229,286

(22) Filed: Sep. 8, 2011

Related U.S. Application Data
(60) Provisional application No. 61/380,983, filed on Sep. 8, 2010.

(51) Int. Cl. C40B 30/00; C40B 40/08; C12Q 1/68; C40B 40/10; C12Q 1/02; G01N 33/53

(52) U.S. Cl. 506/7; 435/2; 506/18; 506/17

(57) ABSTRACT

The present invention is based in part on the identification of a signature marker profile of immune variables to diagnose an immune mediated disease or for prediction of response to an immune mediated disease therapeutic agent. Additionally, the present invention provides methods for the prediction of response to an immune mediated disease therapeutic agent.

### CROSS REFERENCE TO RELATED APPLICATION(S)

This application claims the benefit of priority under 35 U.S.C. §119(e) of U.S. Ser. No. 61/380,983, filed Sep. 8, 2010, the entire content of which is incorporated herein by reference.

### GRANT INFORMATION

This invention was made in part with government support under Grant # AR056273 awarded by NIAMS. The government has certain rights in the invention.

### BACKGROUND OF THE INVENTION

1. Field of the Invention

The present invention relates generally to the field of immune mediated processes and more specifically to the identification of a signature marker profile of immune variables to diagnose a condition or predict response to a therapeutic agent.

2. Background Information

The immune system is a complex matrix of processes which protects the body from various pathogens. The immune system must recognize a wide variety of pathogens, such as viruses and bacteria, and distinguish those from the body's own cells and tissues. When these processes are disrupted immune mediated diseases may result. Immune mediated diseases are conditions which result from abnormal activity of the body's immune system. The immune system may over react or start attacking the body.

Immune mediated diseases can be divided into several categories including immunodeficiency diseases, autoimmune diseases and hypersensitivity diseases. Immunodeficiency diseases occur when part of the immune system is not functioning properly. Autoimmune diseases are the result of the immune system attacking the body instead of pathogens. Hypersensitivity diseases occur when the immune system over reacts and results in damage to the body.

An example of an autoimmune disease is rheumatoid arthritis (RA). RA is a chronic autoimmune disease that leads to inflammation of the joints and surrounding tissues. The disease is characterized by joint inflammation and pain and usually affects joints in a symmetrical fashion. The synovial joints are the area principally attacked, producing an inflammatory response of the synovium, hyperplasia of the synovial cells and excess synovial fluid. The cause of RA is unknown and the disease cannot be cured.

Molecular immunology has provided tools for improved knowledge of the mechanisms contributing to the pathogenesis of immune mediated diseases. The chronic inflammatory nature of many of these diseases is now better understood and many of the mediators and pathways that amplify inflammatory processes and lead to tissue damage have been identified. This progress has translated into clinical practice, for example in RA with the introduction of biologic agents that effectively interfere with the inflammatory cascade by blocking one of its key components. Direct biological interference with cytokines, such as TNFa, and accessory molecules on the surface of immune cells, such as synthetic molecules with CTLA-4 or CD3 on T cells or CD20 on B cells, is increasingly replacing generalized pharmacological immune suppression via induction of immune suppression or immune tolerance.

The clinical effectiveness of immune mediated disease therapeutics varies widely depending on the specific condition and the individual subject receiving the therapeutic agent. Altogether, the level of clinical effectiveness, which specifically for RA leaves approximately half of the patients with an insufficient response, and the fact that there are no objective data available to justify the choice of one biologic versus another, underscore the need to improve the knowledge base for current and future therapeutic decisions. Such knowledge should be grounded on a mechanistic-based approach to identify the effects of these drugs on the immune system and their actual mechanism of action in relationship to clinical responsiveness. Awareness is converging from disparate standpoints regarding the fact that biological therapeutic agents—which were approved based on the assumption that they are specific for the immune pathway with which they were designed to interfere—in reality affect a multiplicity of intersecting mechanisms. There is therefore a need to define mechanisms of action of biologic agents with an approach that takes into account effects on organisms and systems rather then on individual pathways. This is also true of many other types of an immune mediated disease therapeutic agents as well, where a patient is subjected to a several different therapeutic agents before identifying one that elicits a positive response. It is precisely here that a fundamental knowledge gap can be identified.

The overarching strategy of the present invention, is that while the clinical features of patients taking various immune mediated disease therapeutic agents will not be significantly different, mechanistic differences between treatments will be distinguishable, thus defining predictors of efficacy based on immune function. For example, effective therapies may induce low disease activity (LDA) in RA by affecting multiple interdependent immune pathways, including, T-cell regulation, lineage commitment (TH- 1/17, tolerant/anergic) for effector T cells, and dendritic cell function. The present invention utilizes multiple interacting pathways, rather than a single mechanism, to determine susceptibility to treatment on the basis of immunological regulation. Consequently, it is possible to identify immune functions that cluster with a positive response to an immune mediated disease therapeutic. Such signature marker profiles could contribute to defining the mechanism of action of each treatment and provide a knowledge based, logical framework for clinical decisions, including the choice of a given an immune mediated disease therapeutic for treatment of clinically indistinguishable patients. The tangible translational outcomes from this reverse translational itinerary (bedside to bench) relate not only to a better knowledge of mechanism of action of widely used an immune mediated disease therapeutic agents, but also to the potential use of this newly acquired knowledge to support clinical therapeutic decisions.

### SUMMARY OF THE INVENTION

The present invention is based in part on the identification of a signature marker profile of immune variables to diagnose an immune mediated disease or for the prediction of a response to an immune mediated disease therapeutic agent. Additionally, the present invention provides methods for the prediction of response to such a therapeutics.

Accordingly, in one embodiment, the present invention provides a method to diagnose an immune mediated disease or to predict a response to an immune mediated disease therapeutic agent. The method includes isolating proliferating blood mononuclear cells (PBMCs) from a subject diagnosed with or treated for an immune mediated disease, stimulating the PBMCs, identifying subpopulations of PBMCs, comparing data from the PBMC subpopulations to subject diagnosis and response to the therapeutic and selecting a signature marker profile related to a positive diagnosis or response. In one aspect, the PBMC stimulating agent of the method includes an anti-CD3/CD28 antibody, Tetanus toxoid peptides or appropriately selected antigens, known to be contributors to the inflammatory process. In another aspect, the PBMCs are isolated from a subject treated with a biologic agent. In one aspect, the immune mediated disease is rheumatoid arthritis (RA).

In a further aspect, the biologic therapeutic is an antibody which inhibits proteins and processes of the immune system and includes, but is not limited to, adalimumab, etanrecept, infleximab, certolizumab, golimumab, anakinra, rituximab, abatacept, tocilizumab, muronomab, abciximab, daclizumab, basilimab, omalizumab, efalizumab, natalizumab, certolizumab pegol, usterkinumab, belimumab, clenoliximab, keliximab, priliximab, teneliximab, vapaliximab, ibalizumab, aselizumab, apolizumab, benralizumab, cedelizumab, certolizumab pegol, eculizumab, epratuzumab, erlizumab, fontolizumab, mepolizumab, ocrelizumab, pascolizumab, pexelizumab, reslizumab, rontalizumab, rovelizumab, ruplizumab, siplizumab, talizumab, teplizumab, tocilizumab, toralizumab, vedolizumab and visilizumab and any combination of the above. In yet another aspect, the therapeutic is an inhibitor of activated T cells; an inhibitor of B cells; an anti-inflammatory agent; a disease modifying agent; an analgesic agent or any combination thereof.

In a further aspect, the PBMC subpopulation identification includes FACS analysis. The markers identified by FACS analysis include, but is not limited to annexin V, B7-H1, B7-H3, B7-H4, B7-DC, B cells, memory B cells, CCR6, CD1c, CD4, CD8, CD11c, CD14, CD16, CD19, CD25, CD 27, CD28, CD30, CD39, CD40, CD56, CD62L, CD69, CD80, CD86, CD103, CD107, CD123, CD127, CD141, CD200, CTLA-4, CXCR3, Fas, FasL, FoxP3, GARP, GATA-3, GITR, GranzymeA, GranzymeB, HLA-DR, ICOS, IL-1β, IL-2, IL-4, IL-6, IL-10, IL-12, IL-12R, IL17, IL-17A, IL-21, IL-23, INFγ, KOS, memory CD4 cells, monocyte cells, naïve CD4 cells, NK cells, NKT cells, OX40, PD-1, PD-LI/II, Perforin, RORC, STAT-3 phosphorylation, STAT-5 phosphorylation, T-bet, TCR_{gd}, TGFβ, TNFα, T regulatory cells, T effector cells and any combination thereof.

In yet another aspect of the invention, the PBMC subpopulation identification includes a functional assay. The functional assay includes, but is not limited to, a proliferation assay, a suppression assay, confocal microscopy, Western blot, CTV staining, CFSE staining or propidium iodide (PI) assay. The confocal microscopy may further include determining the phosphorylation status of STAT-3 and STAT-5.

In another aspect, PBMC subpopulation identification is by PCR analysis. The PCR analysis includes but is not limited to FoxP3, GATA-3, Granzyme, ICOS, IκBα, IKK, IL-2, IL-4, IL-6, IL-10, IL-12, IL-12R, IL-17A, IL-21, IL-23, INFγ, NFκb pathway, p50, PD-1, Perforin, RelA, RORC, T-bet, TGFβ, TNFα and any combination thereof.

In a further aspect, the PBCM subpopulation includes but is not limited to CD4⁺, CD127⁺ and T effector cells; CD4⁺, CD12T and T regulatory cells; CD4⁺, CD12T, CD25^{low/neg} and nT regulatory cells; CD4⁺, CD127⁻, CD25⁺⁺ and nT regulatory cells; CD4⁺, CD127⁻, CD25⁺⁺, PD-1⁺ and tT regulatory cells; CD14, CD19, CD11c and CD123; CD56⁺, CD3⁻ and NK cells; CD8⁺ and T cells; CD4⁺, CXC3⁺, CCR6⁻, and Th1 cells; CD4⁺, CXC3, CCR6⁺, CD161⁺ and Th17 cells; CD4⁺, CXC3⁺, CCR6⁺, CD161⁺, Th1 cells and Th17 cells; CD8⁻, CD4⁺ and T cells; CD8⁺, CD4⁻ and T cells; CD19⁺ and B cells; CD14⁺ and monocytes; CD4⁺, CD25^{high} and T effector cells; CD4⁺, CD25⁻ and T effector cells; and CD4⁻, CD8⁻, CD 19⁺ and B cells; and any combination thereof.

In another embodiment, the present invention provides a method of monitoring the course of a therapy using an immune mediated disease therapeutic. The method includes isolating PBMCs from a subject previously or currently treated for an immune mediated disease, stimulating the PBMCs, identifying subpopulations of PBMCs, comparing data from the PBMC subpopulations to a subject response to the therapeutic and selecting a signature marker profile related to a positive response to the therapeutic, thereby monitoring the course of therapy.

In a further embodiment, the present invention includes the use of an Immunomics platform to diagnose an immune mediated disease or predict response to an immune mediated disease therapeutic agent. The use includes isolating PBMCs from a subject diagnosed with or treated for an immune mediated disease, stimulating the PBMCs, identifying subpopulations of PBMCs, comparing data being comparing data from the PBMC subpopulations to subject diagnosis or response to the therapeutic and selecting a signature marker profile related to a positive diagnosis or response to the therapeutic agent.

In another embodiment, the present invention provides an epigenetic method to diagnose an immune mediated disease or to predict a response to an immune disease mediated therapeutic agent. The method includes isolating PBMCs from a subject diagnosed with or treated for an immune mediated disease, stimulating the PBMCs, identifying subpopulations of PBMCs, comparing data from the PBMC subpopulations to subject diagnosis or response to therapeutics and selecting a signature marker profile related to a positive diagnosis or response to the therapeutic. In one aspect the signature profile consists of genes or proteins which are up regulated or down regulated in response to therapy. In a further aspect the signature profile consists of methylation patterns of DNA or RNA in response to therapy.

In another aspect, the present invention provides for a protein or gene expression chip. In one aspect, the protein or gene expression chip contains one or more markers of the signature marker profile for predicting a response to a therapeutics.

### BRIEF DESCRIPTION OF THE DRAWINGS

FIG. 1 is a flow chart of the method to identify a signature marker profile for the response to RA therapy using the Immunomics platform.

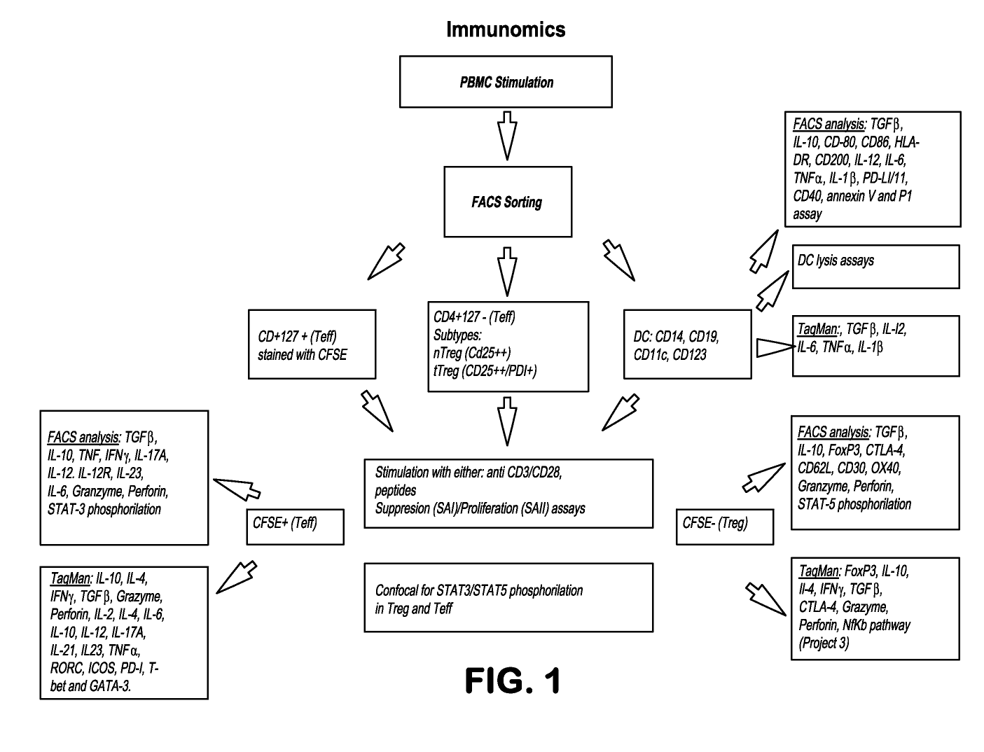

FIG. 2 is a chart showing determination by TaqMan of the expression of IL-4, IL-10 and IL-17 from isolated Teff.

FIG. 3 is a chart showing the determination of suppression activity.

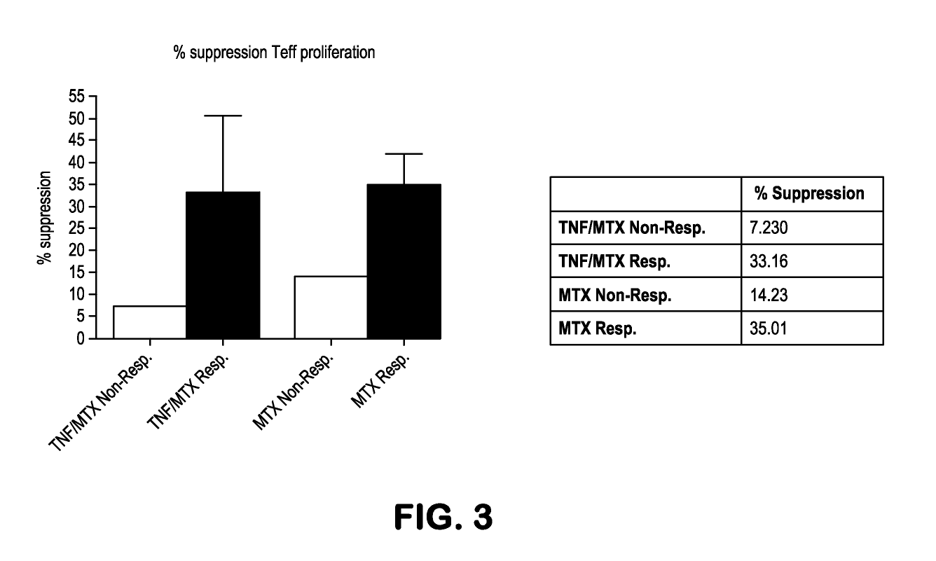

FIG. 4 is a heat map showing immunophenotyping of PBMCs from the TREAT trial were with a panel of markers using a 9-color FACSAria II.

FIG. 5 is a chart showing the staining for CD4, CD25, FoxP3, GITR, Granzyme B and CD69 on PBMCs from T0 and Tend.

### DETAILED DESCRIPTION OF THE INVENTION

The present invention is based in part on the identification of a marker profile of immune variables to diagnose an immune mediated disease or predict a subject's response to an immune mediated disease therapeutic agent. Additionally, the present invention provides methods for the prediction of a subject's response to an immune mediated disease therapeutic agent and the monitoring of a course of treatment with such a therapeutic agent. Furthermore, the present invention provides for protein and gene expression chips containing the signature marker profile.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

As used herein, the term “immune mediated disease” refers to conditions which result from abnormal activity of the body's immune system. Immune mediated diseases can include but are not limited to immunodeficiency diseases, autoimmune diseases and hypersensitiviy diseases. Autoimmune diseases are the result of the immune system attacking the body instead of pathogens. Immunodeficiency diseases occur when part of the immune system is not functioning. Autoimmune diseases are the result of the immune system attacking the body instead of pathogens. Hypersensitivity diseases occur when the immune system over reacts and results in damage to the body.

Immunodeficiency diseases occur when part of the immune system is not functioning properly. Primary immunodeficiency occurs when a person is born lacking a part of the immune system which impairs the immune processes from functioning normally. Acquired immunodeficiency occurs when the body stops producing a component of the immune system. Treatments for immunodeficiency include antibiotics, immunoglobulin therapy, gamma interferon therapy, stem cell transplantation and growth factors.

Hypersensitivity occurs when the immune system over reacts and results in damage to the body. Typically there are four types of hypersensitivity reactions; IgE antibody reaction, Cytokine reaction, immune complex reaction and delayed reaction. Treatments for hypersensitivity include antihistamine medications, corticosteroids, DMARDS, epinephrine injection, immunosuppressive agents, leukotrine receptor antagonists, mast cell stabilizers, and NSAIDS.

Autoimmune diseases are the result of the immune system attacking the body instead of pathogens. There are many types of autoimmune disorders and each uses a different treatment. In general, treatment is directed toward symptom relief, organ preservation and targeting disease mechanisms. Some of the possible treatments include thyroid supplements, insulin, blood transfusion, immunosuppressive agents, NSAIDS, and interferon beta.

Immune mediated diseases include, but are not limited to: allergic bronchopulmonary aspergillosis; allergic rhinitis, autoimmune hemolytic anemia; acanthosis nigricans; allergic contact dermatitis; Addison's disease; atopic dermatitis; alopecia greata; alopecia universalis; amyloidosis; anaphylactoid purpura; anaphylactoid reaction; aplastic anemia; angioedema, hereditary; angioedema, idiopathic; ankylosing spondylitis; arteritis, cranial; arteritis, giant cell; arteritis, Takayasu's; arteritis, temporal; asthma; ataxia-telangiectasia; autoimmune oophoritis; autoimmune orchitis; autoimmune polyendocrine failure; Behcet's disease; Berger's disease; Buerger's disease; bronchitis; bullous pemphigus; candidiasis, chronic mucocutaneous; Caplan's syndrome; post-myocardial infarction syndrome; post-pericardiotomy syndrome; carditis; celiac sprue; Chagas's disease; Chediak-Higashi syndrome; Churg-Strauss disease; Cogan's syndrome; cold agglutinin disease; CREST syndrome; Crohn's disease; cryoglobulinemia; cryptogenic fibrosing alveolitis; dermatitis herpetifomis; dermatomyositis; diabetes mellitus; Diamond-Blackfan syndrome; DiGeorge syndrome; discoid lupus erythematosus; eosinophilic fasciitis; episcleritis; drythema elevatum diutinum; erythema marginatum; erythema multiforme; erythema nodosum; Familial Mediterranean fever; Felty's syndrome; pulmonary fibrosis; glomerulonephritis, anaphylactoid; glomerulonephritis, autoimmune; glomerulonephritis, post-streptococcal; glomerulonephritis, post-transplantation; glomerulopathy, membranous; Goodpasture's syndrome; granulocytopenia, immune-mediated; granuloma annulare; granulomatosis, allergic; granulomatous myositis; Grave's disease; Hashimoto's thyroiditis; hemolytic disease of the newborn; hemochromatosis, idiopathic; Henoch-Schoenlein purpura; hepatitis, chronic active and chronic progressive; histiocytosis X; hypereosinophilia syndrome; idiopathic thrombocytopenic purpura; Job's syndrome; juvenile dermatomyositis; juvenile rheumatoid arthritis (Juvenile chronic arthritis); Kawasaki's disease; keratitis; keratoconjunctivitis sicca; Landry-Guillain-Barre-Strohl syndrome; leprosy, lepromatous; Loeffler's syndrome; lupus; Lyell's syndrome; lyme disease; lymphomatoid granulomatosis; mastocytosis, systemic; mixed connective tissue disease; mononeuritis multiplex; Muckle-Wells syndrome; mucocutaneous lymph node syndrome; mucocutaneous lymph node syndrome; multicentric reticulohistiocytosis; multiple sclerosis; myasthenia gravis; mycosis fungoides; necrotizing vasculitis, systemic; nephrotic syndrome; overlap syndrome; panniculitis; paroxysmal cold hemoglobinuria; paroxysmal nocturnal hemoglobinuria; pemphigoid; pemphigus; pemphigus erythematosus; pemphigus foliaceus; pemphigus vulgaris; pigeon breeder's disease; pneumonitis, hypersensitivity; polyarteritis nodosa; polymyalgia rheumatic; polymyositis; polyneuritis, idiopathic; portuguese familial polyneuropathies; pre-eclampsia/eclampsia; primary biliary cirrhosis; progressive systemic sclerosis (scleroderma); psoriasis; psoriatic arthritis; pulmonary alveolar proteinosis; pulmonary fibrosis, Raynaud's phenomenon/syndrome; Reidel's thyroiditis; Reiter's syndrome, relapsing polychrondritis; rheumatic fever; rheumatoid arthritis (RA); sarcoidosis; scleritis; sclerosing cholangitis; serum sickness; Sezary syndrome; Sjogren's syndrome; Stevens-Johnson syndrome; Still's disease; subacute sclerosing panencephalitis; sympathetic ophthalmia; systemic lupus erythematosus; yransplant rejection; ulcerative colitis; undifferentiated connective tissue disease; urticaria, chronic; urticaria, cold; uveitis; vitiligo; Weber-Christian disease; Wegener's granulomatosis, or Wiskott-Aldrich syndrome. Immune mediated disease may also include transplant organ rejection such as heart, kidney, liver, skin, pancreatic islet cells or bone marrow rejection. Immune mediated disease may also include graft-vs-host disease in a subject.

Additionally, immune mediated diseases may include pulmonary fibrosis or fibrotic disease. Some examples of pulmonary fibrosis include: pulmonary fibrosis secondary to adult respiratory distress syndrome, drug-induced pulmonary fibrosis, idiopathic pulmonary fibrosis, or hypersensitivity pneumonitis. Some examples of fibrotic diseases include: Hepatitis-C; Hepatitis-B; cirrhosis; cirrhosis of the liver secondary to a toxic insult; cirrhosis of the liver secondary to drugs; cirrhosis of the liver secondary to a viral infection; and cirrhosis of the liver secondary to an autoimmune disease.

Further, immune mediated disease may include, but are not limited to, gastrointestinal disease including: esophageal dysmotility, inflammatory bowel disease (including Crohn's disease and ulcerative colitis), gastritis, collagenous colitis (including lymphocytic colitis and microscopic colitis), coeliac disease (also called gluten enteropathy, coeliac sprue, or gluten intolerance), and scleroderma. In another aspect immune mediated disease may include: atherosclerosis, renal artery disease, lymphedema, ischemic disorders, reperfusion injury, collagen vascular/immune complex diseases such as systemic lupus erythematosis or cryoglobulinemia.

Further, autoimmune diseases may include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g. Type I diabetes mellitus or insulin dependent diabetes mellitis); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia, Myasthenia gravis, systemic lupus erythematosus, Graves' disease, idiopathic thrombocytopenia purpura, hemolytic anemia, diabetes mellitus, inflammatory bowel disease, Crohn's disease, multiple sclerosis, psoriasis, drug-induced autoimmune diseases, or drug-induced lupus. In certain embodiments, the autoimmune response is associated with or derived from systemic lupus erythematosus, infectious disease, Reiter's syndrome, spondyloarthritis, Lyme disease, HIV infection, syphilis, or tuberculosis etc.

Additionally, immune mediated disease also includes traumatic brain injury and depression.

As used herein, the terms “rheumatoid arthritis” or “RA” refers to an autoimmune disease which primarily affects joints. RA includes, but is not limited to, adult RA, juvenile iodopathic arthritis, juvenile rheumatoid arthritis and juvenile chronic arthritis.

RA is an example of an autoimmune mediated disease and is incurable, once bone or joint damage occurs it is usually irreversible. It is therefore, important that RA treatment begin soon after the onset of symptoms to delay or halt disease progression and prevent irreversible damage.

As used herein, the term “an immune mediated disease therapeutic” or “immune mediated disease therapeutic agent” refers to any therapeutic agent used to treat an immune mediated disease.

DMARDs are a broad class of agents which are know as disease modifying anti-rheumatic drugs, which are used in a variety of immune mediated diseases. DMARDS include azathioprine, ciclosporin, D-penicillamine. Gold salts, hydroxychloroquine, leflunomide, methotrexate, minocycline, sulfasalazine and cyclophosphamide. Treatments encompassing combinations of DMARDS are common.

Analgesics and anti-inflammatory agents are also frequently used in treating immune mediated diseases. Typical analgesics include acetaminophen, opiates, diproqualone, and topical lidocaine. Typical anti-inflammatory agents include gluccocorticoids and non-steroidal anti-inflammatory agents (NSAIDS). Glucocorticoids include hydrocortisone, prednisone, prednisolone, methylprednisolone, dexamethasone, betamethasone, triamcinolone, beclometasone, fludrocortisone acetate, deoxycorticosterone acetate, and aldosterone. Typical NSAIDS include ibuprofen, maproxen, meloxicam, etodolac, nabumetone, sulindac, tolementin, choline magnesium salicylate, diclofenac, diflusinal, indomethicin, ketoprofen, oxaprozin and piroxicam.

Biologic agents are medicinal products such as a vaccine, blood or blood component, allergenic, somatic cell, gene therapy, tissue, recombinant therapeutic protein, or living cells that are used as therapeutics. Unlike other drugs which are chemically synthesized, biologic agents are made through biologic processes. For example, therapeutic antibodies are produced in live cells and secreted into the surrounding media. Biologic agents have been very effective in alleviating symptoms of arthritis. Many of these agents affect the immune system and t have some significant side effects and are therefore usually used only after other treatments have failed. Side effects that can occur with these agents include injection site reactions (rash, burning), tuberculosis, bacterial infections, allergic reactions and progressive multifocal leukoencephalopathy, which can be fatal. It is therefore imperative that patients on an immune mediated disease therapeutic agent which is a biologic be monitored closely and ideally patients would be screened prior to treatment to determine which biologic agent would be effective.

Biologic agents used as immune mediated disease therapeutics inhibit various different immune system related proteins and processes. Adalimumab, etanrecept, infleximab, certolizumab, and golimumab inhibit TNF. Anakinra targets II-1. Rituximab targets CD20 on B cells. Abatacept acts as a T cell costimulation blocker. Tocilizumab targets IL-6. Other examples of biologic immune mediated disease therapeutics include, but is not limited to, muronomab, abciximab, daclizumab, basilimab, omalizumab, efalizumab, natalizumab, certolizumab pegol, usterkinumab, belimumab, clenoliximab, keliximab, priliximab, teneliximab, vapaliximab, ibalizumab, aselizumab, apolizumab, benralizumab, cedelizumab, certolizumab pegol, eculizumab, epratuzumab, erlizumab, fontolizumab, mepolizumab, ocrelizumab, pascolizumab, pexelizumab, reslizumab, rontalizumab, rovelizumab, ruplizumab, siplizumab, talizumab, teplizumab, tocilizumab, toralizumab, vedolizumab and visilizumab.

In one embodiment, the present invention provides a method for diagnosis of an immune mediated disease or predicting a response to an immune mediated disease therapeutic agent. The method includes isolating PBMCs from a subject diagnosed with or treated for an immune mediated disease, stimulating the PBMCs, determining subpopulations of PBMCs, comparing data from the PBMC subpopulations to a subject diagnosis or response to therapeutic agent and selecting a marker profile related to a positive diagnosis or response to therapeutic.

As used herein, the term “PBMC” refers to peripheral blood mononuclear cell. A PBMC is any blood cell having a round nucleus. These cells are critical components of the immune system to fight infection. PBMCs include lymphocytes, monocytes and macrophages. Lymphocytes include T cells, B cells and natural killer (NK) cells. PBMCs can be stimulated so that T cells proliferate. Agents that can stimulate PBMCs and therefore T cell proliferation, include anti CD3/CD28 antibody, Tetanus toxoid peptides or appropriately selected antigens, known to be contributors to the inflammatory process.

T cells are integral to cell mediated immunity and all express CD4. There are several types of T cells: helper T cells (T_{H} cells), cytotoxic T cells (T_{c} cells), memory T cells, regulatory T cells (T_{reg} cells), effector T cells (T_{E} cells) and natural killer cells (NKT cells). T_{H} cells assist other T cells in immunologic presses, such as maturation of B cells and activation of T_{C} cells. T_{H} cells are activated by MCH class II molecules expressed on the surface of antigen presenting cells (APCs). T_{reg} cells are responsible for shutting down T cell mediated immunity and express CD4, CD25 and FoxP3. T_{E} cells contribute to the fundamental reactions of adaptive immunity.

PBCMs are isolated from the blood typically using ficoll which traps monocytes and lymphocytes in a buffy coat which forms underneath plasma in a tube. Another way to isolate PBMCs is to extract the cells from whole blood using hypotonic lysis which will preferentially lyse red blood cells.

An aspect of the present invention includes the identification of a subpopulation of PBMCs from patients that had a positive diagnosis for an immune mediated disease or positive response to an immune mediated disease therapeutic agent. This subpopulation of PBMCs will have differences in immune variables compared to those patients that didn't have a positive diagnosis or response to therapy. This difference in immune variables provides a way to identify patients who will also have a positive diagnosis or response to the therapeutic and is therefore important to help with clinical decisions. Given the severity of the side effects associate with treatment using some immune mediated disease therapeutics, such information is invaluable. There are several methods that can be used alone or in combination to identify this subpopulation including FACS analysis, functional assays and PCR analysis. Subpopulations of the instant invention may include, but are not limited to, CD4⁺, CD127⁺ and T effector cells; CD4⁺, CD127⁻ and T regulatory cells; CD4⁺, CD127⁻, CD25^{low/neg} and nT regulatory cells; CD4⁺, CD127⁻, CD25⁺⁺ and nT regulatory cells; CD4⁺, CD127⁻, CD25⁺⁺, PD-1⁺ and tT regulatory cells; CD14, CD19, CD11c and CD123; CD56⁺, CD3⁻ and NK cells; CD8⁺ and T cells; CD4⁺, CXC3⁺, CCR6⁻, and Th1 cells; CD4⁺, CXC3, CCR6⁺, CD161⁺ and Th17 cells; CD4⁺, CXC3⁺, CCR6⁺, CD161⁺, Th1 cells and Th17 cells; CD8⁻, CD4⁺ and T cells; CD8⁺, CD4⁻ and T cells; CD19⁺ and B cells; CD14⁺ and monocytes; CD4⁺, CD25^{high} and T effector cells; CD4⁺, CD25⁻ and T effector cells; and CD4⁻, CD8⁻, CD 19⁺ and B cells; and any combination thereof.

In one aspect of the invention, FACS analysis may be used to sort cells based upon the specific light scattering and fluorescent characteristics of each cell. Cells are fluorescently labeled for different characteristics (such as expression of certain proteins) and the FACS apparatus can separate the cells into distinct populations. In the present invention FACS analysis may be used for the isolation of different types of T cells through specific cell surface markers or as a means of identifying the subpopulation of PBMCs from patients that responded to therapy based on a unique combination of cell surface markers. For example the cell surface markers may include, but are not limited to, annexin V, B7-H1, B7-H3, B7-H4, B7-DC, B cells, memory B cells, CCR6, CD1c, CD4, CD8, CD11c, CD14, CD16, CD19, CD25, CD 27, CD28, CD30, CD39, CD40, CD56, CD62L, CD69, CD80, CD86, CD103, CD107, CD123, CD127, CD141, CD200, CTLA-4, CXCR3, Fas, FasL, FoxP3, GARP, GATA-3, GITR, GranzymeA, GranzymeB, HLA-DR, ICOS, IL-1β, IL-2, IL-4, IL-6, IL-10, IL-12, IL-12R, IL17, IL-17A, IL-21, IL-23, INFγ, KOS, memory CD4 cells, monocyte cells, naïve CD4 cells, NK cells, NKT cells, OX40, PD-1, PD-LI/II, Perforin, RORC, STAT-3 phosphorylation, STAT-5 phosphorylation, T-bet, TCR_{gd}, TGFβ, TNFα, T regulatory cells, T effector cells and any combination thereof.

In another aspect of the invention, the identification of the subpopulation of PBMCs may include the use of functional assays. Functional assays are assays that measure an activity of the cell. Functional assays include proliferation assays and suppression assays which measure the growth or lack thereof of a cell population. Other functional assays include staining with CTV (Cell Trace Violet) to identify T_{reg} cells and carboxyfluorescein diacetate succinimidyl ester (CFSE) to identify T_{E} cells. Another functional assay is confocal microscopy which may be used to determine the phosphorylation status of STAT3 and STAT5. Additionally, western blot analysis can be used to detect the presence or absence of proteins.

In a further aspect, PCR analysis may also be used for the identification of the subpopulation of PBMCs. PCR analysis can be used to identify genes that are expressed and to quantify and identify changes in expression of genes. PCR markers of the present invention include, but are not limited to, FoxP3, GATA-3, Granzyme, ICOS, IκBα, IKK, IL-2, IL-4, IL-6, IL-10, IL-12, IL-12R, IL-17A, IL-21, IL-23, INFγ, NFκb pathway, p50, PD-1, Perforin, RelA, RORC, T-bet, TGFβ, TNFα and any combination thereof.

As used herein, the term “signature marker profile” refers to a set of immune variables identified from PBMCs of patients which have been diagnosed with an immune mediated disease or have responded to an immune mediated disease therapeutic agent. This signature marker profile may then be used to diagnose an immune mediated disease or to screen patients and predict whether these patients would have a positive response to an immune mediated disease therapeutic agent. Pre-screening patients would aid in clinical decisions and reduce any unnecessary side effects by ensuring patients are only given therapeutics for which they are predicted to have a positive response. The signature marker profile may also be used to monitor patient currently undergoing therapy to ensure the immune mediated disease therapeutic agent is working. The signature profile may vary depending on the specific therapeutic agent which is used.

In another embodiment, the present invention provides a method of monitoring the course of a therapy using an immune mediated disease therapeutic agent. The method includes isolating PBMCs from a subject diagnosed with or treated for an immune mediated disease, stimulating the PBMCs, determining subpopulations of PBMCs, comparing data from the PBMC subpopulations to a subject response to the therapeutic agent and selecting a marker profile related to a positive response to the therapeutic agent.

In a further embodiment, the present invention provides a method of predicting response to an immune mediated disease therapeutic agent. The method including obtaining a blood sample from a subject in need of therapy, analyzing the blood sample for the signature marker profile for positive response to the therapeutic agent and identifying the profile markers present in the blood sample. In one aspect of the method, the blood sample is analyzed by methods including a protein expression chip; a gene expression chip, protein expression, detection of phosphorylation, phosphorescence or luminescence detection, immunobeads or a combination thereof.

In one aspect, the present invention provides a method for the diagnosis of traumatic brain injury. The method including obtaining a blood sample from a subject in need of such a diagnosis, analyzing the blood sample for the signature marker profile identified for traumatic brain injury and identifying the profile markers present in the blood sample. In one aspect of the method, the blood sample is analyzed by methods including a protein expression chip; a gene expression chip, protein expression, detection of phosphorylation, phosphorescence or luminescence detection, immunobeads or a combination thereof.

In another aspect, the present invention provides a method to distinguish between Type I and Type II diabetes. The method including obtaining a blood sample from a subject suspected of having Type I or Type II diabetes, analyzing the blood sample for the signature marker profile for Type I and Type II diabetes and identifying the profile markers present in the blood sample. In one aspect of the method, the blood sample is analyzed by methods including a protein expression chip; a gene expression chip, protein expression, detection of phosphorylation, phosphorescence or luminescence detection, immunobeads or a combination thereof.

In a further aspect, the present invention provides a method to diagnosis and treat depression. The method including obtaining a blood sample from a subject in need of a diagnosis or therapy for depression, analyzing the blood sample for the signature marker profile identified for depression and identifying the profile markers present in the blood sample. In one aspect of the method, the blood sample is analyzed by methods including a protein expression chip; a gene expression chip, protein expression, detection of phosphorylation, phosphorescence or luminescence detection, immunobeads or a combination thereof.

In another aspect, the present invention provides for a protein or gene expression chip containing the signature marker profile. Protein and gene expression chips are used to detect and measure levels of either proteins, DNA or RNA. The protein or gene expression chips of the present invention may be used to confirm a diagnosis and/or predict which patients will respond to an immune mediated disease therapeutic and/or to monitor a patient undergoing a course of treatment with such a therapeutic.

In a further aspect, the present invention includes the use of an Immunomics platform to support diagnose an immune mediated disease or predict response to an immune mediated disease therapeutic agent. The use includes isolating PBMCs from a subject treated for an immune mediated disease, stimulating the PBMCs, determining subpopulations of PBMCs, comparing data comparing data from the PBMC subpopulations to a subject diagnosis or response to the therapeutic and selecting a marker profile related to a positive diagnosis or response to the therapeutic.

In another embodiment of the present invention Immunomics platform is used to diagnose subjects with immune mediated disease and to predict which subjects will respond to an immune mediated disease therapeutic agent and/or to monitor a patient undergoing a course of treatment with such a therapeutic agent. The Immunomics platform is the integration of several technologies into a single platform. These technologies include, but are not limited to, FACS analysis, functional assays, PCR analysis, a protein expression chip; a gene expression chip, protein expression, detection of phosphorylation, phosphorescence or luminescence detection, immunobeads or a combination thereof.

In another embodiment, the present invention provides an epigenetic method to support a diagnosis diagnose an immune mediated disease or for predicting a response to an immune mediated disease therapeutic agent. The method includes isolating proliferating blood mononuclear cells (PBMCs) from a subject diagnosed with or treated for an immune mediated disease, stimulating the PBMCs, identifying subpopulations of PBMCs, comparing data from the PBMC subpopulations to subject diagnosis or response to therapeutic agent and selecting a signature marker profile related to a positive diagnosis or response to the therapeutic. In one aspect the signature profile consists of genes or proteins which are up regulated or down regulated in response to therapy. In a further aspect the signature profile consists of methylation patterns of DNA or RNA in response to therapy.

In another aspect, the present invention provides for a protein or gene expression chip. In one aspect, the protein or gene expression chip contains the signature marker profile for predicting response to an immune mediated disease therapeutic agent. In one aspect the signature profile consists of genes or proteins which are up regulated or down regulated in response to therapy. In a further aspect the signature profile consists of methylation patterns of DNA or RNA in response to therapy.

The following examples are intended to illustrate but not limit the invention.

### Example 1

The aim of this study is to identify clusters of immune pathways characteristic of responsiveness that are specific to treatment. The data below pertain to a preliminary study conducted in patients with RA, (n=8), who were first treated with methotrexate (MTX) and stratified based on clinical responsiveness based on Disease Activity Score of 3.2 (DAS3.2). Non-responders (DAS>5.1) were given an anti-TNFa (herein TNF or ETN) in addition to MTX. We show as summary a color coded table. We clustered individual immune functions according to cut off values identified in preliminary studies (25% change compared with baseline at active disease). The Table depicts the differences for patients who are all responders and therefore otherwise clinically indistinguishable (DAS 23.2).

PBMCs from each patient were incubated for 48 hours with plate bound anti-CD3/CD28. Non-adherent cells were collected to enrich T cells by pan T-cell MACS. T cells were FACS sorted on CD4+CD127+(Teff) and CD4+CD25+CD127−(nTreg), and CD4+CD25+PD1+CD127−(PD1+Treg). Aliquots of all sorted cells were collected for TaqMan. The remainder of the APCs and Teff were cultured with isolated Treg. CFSE was added to the Teff cells and cultured for a total of 4 days with anti-CD3/CD28.

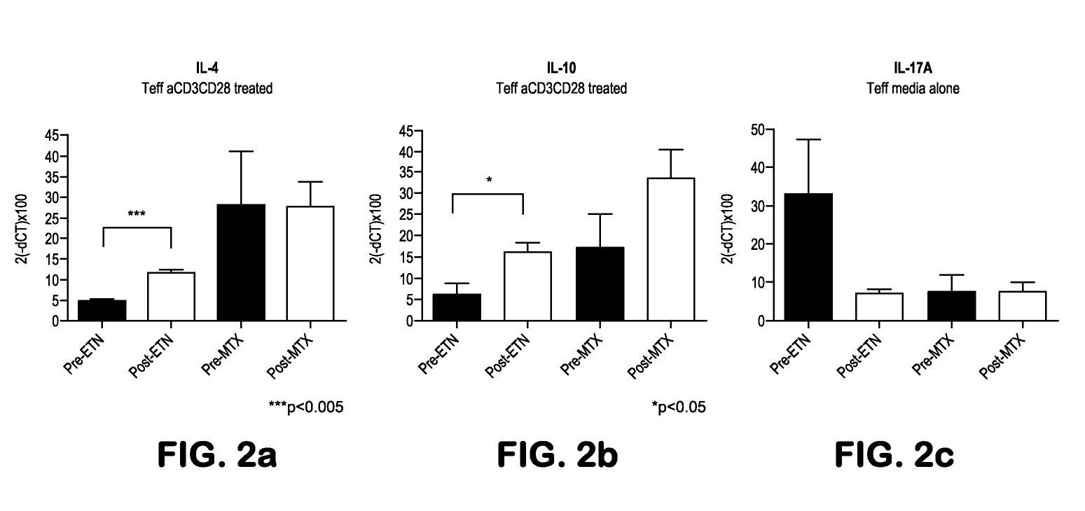

The treatment-induced changes in effector T cells as demonstrated by IL-4 and IL-10 levels which were significantly restored in MTX failures after successful combination therapy (FIG. 2). Basal levels are profoundly different between the two groups at TO. This applies also to the pro-inflammatory cytokine IL-17. Levels of IL-17 were elevated at TO in MTX failures; whereas successful ETN/MTX treatment leads to a reduction of IL-17 expression.

Regulatory T cell suppression is restored by treatment. In both MTX-alone and anti-TNFa/MTX treatment, suppressive capability of Treg to suppress effector proliferation is enhanced when clinical response is seen.

Taken together, these preliminary studies, which need to be sufficiently powered and then validated, suggest the existence of a circuit of active immune tolerization, which seems relevant to clinical improvement. Importantly, this preliminary work also suggests the presence of differences in immune function between the group of MTX failures and MTX

TABLE 1
Visual representation of levels of gene expression for Treg, Teff and APC
pertaining to clinical responders to MTX-ETN (labeled TNF) and to MTX alone.

| | APC | | | | Tₑ Cells | | | |
|---|---|---|---|---|---|---|---|---|
| | | up or | | up or | | up or | | up or |
| | | down | MTX | down | TNF | down | MTX | down |
| | TNF resp | regulated | resp | regulated | resp | regulated | resp | regulated |
| Tbet | | | | | −5.2% | | −17.1% | |
| GATA-3 | | | | | 25.9% | up | −21.6% | |
| | | | | | | regulated | | |
| IL-10 | −0.05% | | +28.6% | up | +28.6% | up | +15.2% | no |
| | | | | regulated | | regulated | | change |
| IL-17A | | | | | −25.1% | down | +0.3% | no |
| | | | | | | regulated | | change |
| IL-21 | | NA | | NA | −71.3% | down | | NA |
| | | | | | | regulated | | |
| TNFα | | NA | | NA | −401% | | 2.3% | |
| INFγ | +40.7% | up | +219.9% | up | 41.0% | down | +1.7% | |
| | | regulated | | regulated | | regulated | | |
| TGFβ | +18.0% | | +31.1% | up | 6.3% | | +150.8% | up |
| | | | | regulated | | | | regulated |
| PD-1 | | | | | 24.1% | | +5.6% | |
| FAS-L | | | | | +29.7% | up | 20.5% | |
| | | | | | | regulated | | |
| ICOS | | | | | −23.8% | | −7.2% | |
| B7-H1 | +94.0% | up | +106.4% | up | | | | |
| | | regulated | | regulated | | | | |
| B7-H2 | +1681.2% | up | +98.4% | up | | | | |
| | | regulated | | regulated | | | | |
| B7-H3 | 70.0% | up | | NA | | | | |
| | | regulated | | | | | | |
| B7-H4 | +1014.2% | up | +118.6% | up | | | | |
| | | regulated | | regulated | | | | |
| B7-DC | | NA | | NA | | | | |
| CD80 | +66.7% | up | | NA | | | | |
| | | regulated | | | | | | |
| CD86 | +16.7% | | | NA | | | | |
| FAS-L | −92.0% | down | +83.1% | up | | | | |
| | | regulated | | regulated | | | | |
| | nTreg | | | | PD1 + Treg | | | |
| | TNF | | MTX | | TNF | | MTX | |
| FoxP3 | +22.7% | no | 439.0% | up | −73.3% | down | +124.1% | up |
| | | change | | regulated | | regulated | | regulated |
| PD1 | +54.4% | up | +474.2% | up | −80.0% | down | 290.3% | up |
| | | regulated | | regulated | | regulated | | regulated |
| CTLA4 | 20.8% | no | +684.0% | up | −67.6% | down | +161.0% | up |
| | | change | | regulated | | regulated | | regulated |
| Granzyme B | −37.7% | down | +393.7% | up | −29.9% | down | +213.8% | up |
| | | regulated | | regulated | | regulated | | regulated |
| Perforin | +96.6% | up | | NA | +91.3% | up | +288.7% | up |
| | | regulated | | | | regulated | | regulated |
| TGFβ | +16.1% | no | +69.3% | up | −2.6% | no | +254.4% | up |
| | | change | | regulated | | change | | regulated |
| IL-10 | +9.4% | no | | NA | −78.8% | down | 168.8% | up |
| | | change | | | | regulated | | regulated |
| INFγ | −41.1% | down | +99.6% | up | +35.5% | up | +355.0% | up |
| | | regulated | | regulated | | regulated | | regulated |

Values are compared to initiation of the treatment and represent the percent change in expression of a given gene as measured by Taqman.
Unpaired t test was used for statistical analysis.

### Example 2

The TREAT study is a recently concluded clinical trial, which tested the difference between two treatment (MTX+TNFa antibody+prednisone+NSAIDS and MTX+prednisone+NSAIDS) arms in patients with active MA who achieved inactive disease (ID). Samples from most patients were collected and stored frozen at two time points: Initiation of treatment (T0) and end (Tend). The data below are from a set of 10 patients: 7 with ID and 3 with no ID in the same treatment arm.

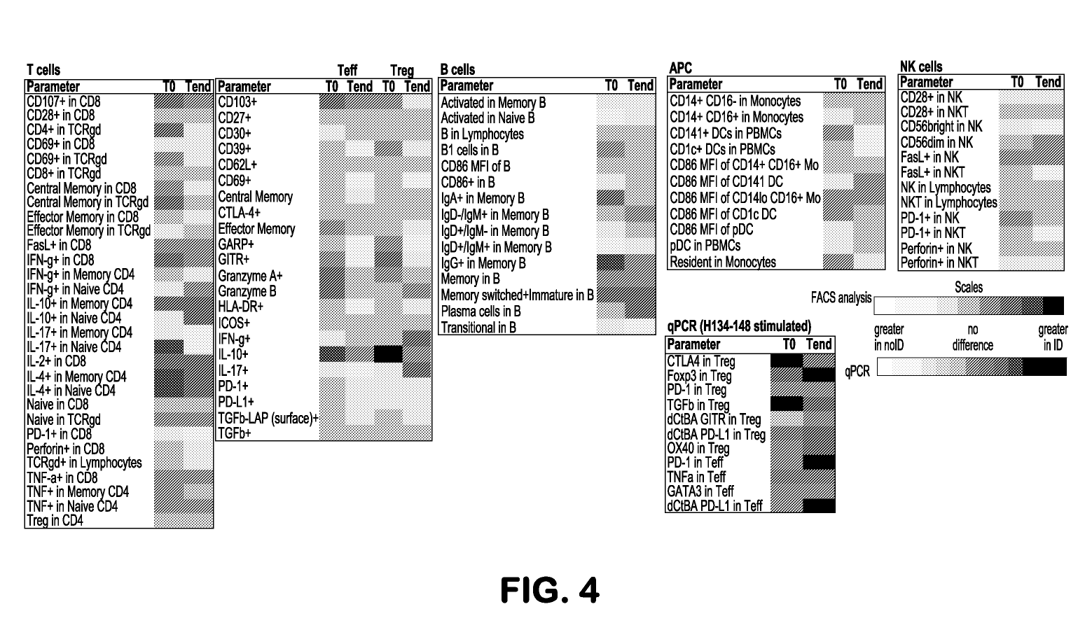

PBMCs from the TREAT trial were thawed and a fraction immunophenotyped with a panel of markers using a 9-color FACSAria II. Any residual PBMCs were sorted for CD4⁺CD25^{low/neg}CD127⁺ effector T cells (Teff), CD4⁺CD25^{high}CD127^{low/neg} regulatory T cells (Treg), CD19+B cells, CD56+CD3−NK cells and CD8+T cells for functional assays. Teff and Treg were differentially labeled with vital dyes (CFSE and CTV respectively) to discriminate them, then stimulated with H134-148 peptide in the presence of autologous APC. After 2 days, Teff and Treg were sorted for qPCR analysis of gene expression. CD8+T cells were stimulated for 6 hours with anti-CD3 crosslinked by P815 cells in the presence of CD107a antibody, monensin and brefeldin A. At the end of the experiment, the percentage of degranulating CD8+T cells was assessed by the expression of CD107a. In addition, an intracellular staining for IFN-γ, TNF-α and IL-2 was performed on those cells. Other functional assays, not shown here, include proliferation and suppression assays of CD4+T cells, activation of B cells via B cell-receptor, CD40 and/or Toll-like receptors to determine proliferation and cytokine production, and NK cell degranulation in response to K562 targets. Data were stratified by time point and disease activity. Differences in each parameter between inactive or not inactive disease within a time point were normalized then color-coded in a heat map (FIG. 4). The heat map represents changes in each parameter between patients who ended therapy with inactive disease versus those who still showed active disease (FIG. 4). Changes are calculated as differences of values normalized in a 0 to 100% scale (where 100% is the maximum value in the whole dataset for each parameter) for flow cytometry data or as Log2 of relative expression for qPCR data.

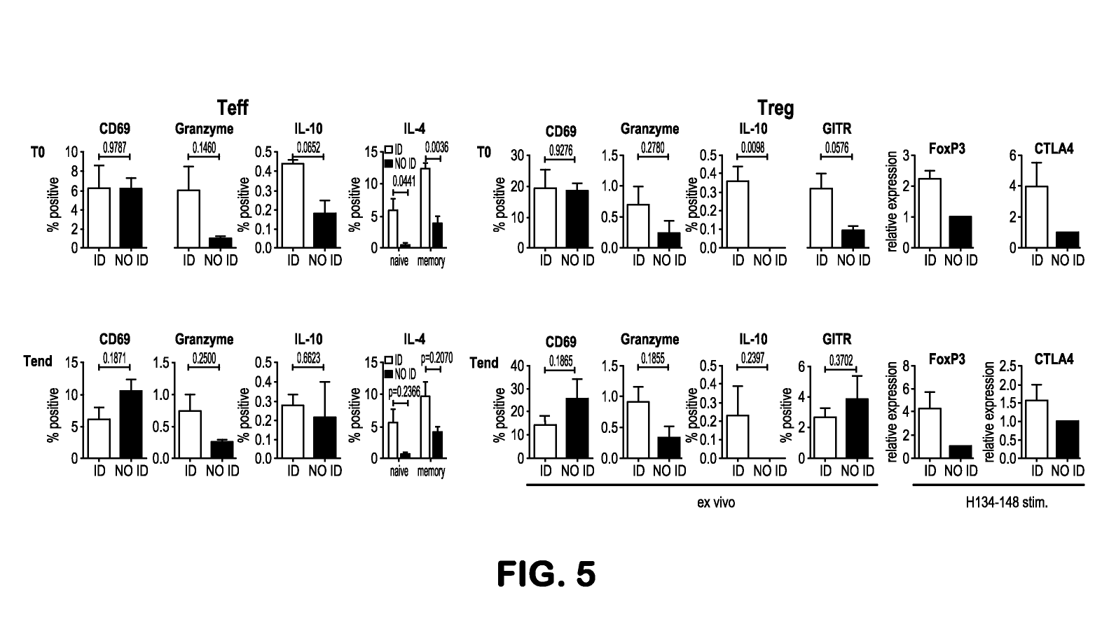

Thawed PBMC from T0 and Tend were stained for CD4, CD25, FoxP3, GITR, Granzyme B and CD69 without manipulation (n=6 to 7 for ID, n=3 for NO ID). Analyses were performed on gated CD4+CD25highFoxP3+(Treg) or CD4⁺CD25^{low/neg} (Teff) cells. A portion of the cells were stimulated with PMA/ionomycin for 6 hr (in the presence of brefeldin A for the last 4 hr) before staining for intracellular IL-10 and IL-4 (n=2 to 3 for ID, n=3 for NO ID). In functional assays, PBMC were sorted into CD4⁺CD25⁻CD127⁺ Teff and CD4⁺CD25^{high}CD127^{low/neg} Treg. Teff were labeled with CFSE, Treg with CTV, a CFSE analog. T cells were then co-cultured with APC presenting H134-148. After 2 days, cells were sorted based on differential labeling (CTV for Treg and CFSE for Teff) to perform qPCR for CTLA-4 and FoxP3 expression (n=2 for ID, n=1 for NO ID), shown in relative scale using the “NO ID” condition as reference (FIG. 5). ID=PBMC from patients who reached inactive disease state at the end of the TREAT clinical trial; NO ID=PBMC from patients who did not reach inactive disease state at the end of the trial.

Heat map of immune functions tested in TREAT evidences clustering associated with responsiveness to therapy. For visualization purposes only, we organized the 132 immune variables tested into a heat map (FIG. 4). Even within the limited sample size tested to date, the relevance and feasibility of the approach in identifying individual and clustered immune functions associated with responsiveness to therapy are evident

From the heat map shown above (FIG. 4) a cluster of phenotypical and functional markers that characterize Teff and Treg were identified. Importantly, these data are obtained both ex vivo (i.e. directly by FACS from unmanipulated PBMC, as in SA 1) and upon stimulation with H134-148, an HSP-derived peptide (as in SA 2). FIG. 5 shows that baseline (T0) expression of IL-10, Granzyme B, and GITR, and CTLA-4 (upon stimulation in vitro with H134-148) are elevated in patients who will achieve ID. Teff function appears skewed toward a more tolerogenic phenotype at T0 in future ID patients as well. At Tend, Treg in patients with ID showed higher values of IL-10, Granzyme and FoxP3 (upon stimulation with H134-148). Teff in ID patients showed a lower proportion of activated cells (CD69+) and higher values of Granzyme B and IL-4.

The preliminary data shown provide proof of feasibility with respect to 1) collection, preparation, shipping and processing of high quality biologic samples as part of a clinical trial performed in pediatric rheumatology sites adequacy of cell numbers in the biologic samples to perform the assays: 3) adequate power to perform the biologic assays; 4) most importantly these data give a clear indication that distilling a cluster of immune responses relevant to a given clinical condition is feasible.

### Example 3

Patient samples are collected from both a JIA study and a RA study. Both studies have two treatment arms: MTX+TNFa antibody+prednisone+NSAIDS and MTX+prednisone+NSAIDS. Samples are collected from time points T0 and Tend of both arms of both studies. Patients are stratified into groups based on response to treatment. The patient samples are analyzed using a gene chip for example. Genes which are expressed and genes which are not expressed are identified for the groups that responded to treatment. A signature marker profile is then identified based on the gene expression pattern and/or the DNA or RNA methylation pattern.

Although the invention has been described with reference to the above example, it will be understood that modifications and variations are encompassed within the spirit and scope of the invention. Accordingly, the invention is limited only by the following claims.

What is claimed is:

1. A method for predicting a response to an immune mediated disease therapeutic agent comprising:
a) isolating proliferating blood mononuclear cells (PBMCs) from a subject treated for an immune mediated disease;
b) stimulating the PBMCs;
c) identifying subpopulations of PBMCs;
d) comparing data from step (c) to subject response to the therapeutic; and
e) selecting a marker profile related to a positive response to the an immune mediated disease therapeutic agent.

2. The method of claim 1, wherein the therapeutic agent is a biologic agent.

3. The method of claim 2, wherein the biologic agent is an antibody.

4. The method of claim 3, wherein the antibody is selected from the group consisting of adalimumab, etanrecept, infleximab, certolizumab, golimumab, anakinra, rituximab, abatacept, tocilizumab, muronomab, abciximab, daclizumab, basilimab, omalizumab, efalizumab, natalizumab, certolizumab pegol, usterkinumab, belimumab, clenoliximab, keliximab, priliximab, teneliximab, vapaliximab, ibalizumab, aselizumab, apolizumab, benralizumab, cedelizumab, certolizumab pegol, eculizumab, epratuzumab, erlizumab, fontolizumab, mepolizumab, ocrelizumab, pascolizumab, pexelizumab, reslizumab, rontalizumab, rovelizumab, ruplizumab, siplizumab, talizumab, teplizumab, tocilizumab, toralizumab, vedolizumab and visilizumab and any combination of the above.

5. The method of claim 1, wherein the immune mediated disease is selected from the group consisting of arthritis, atherosclerosis, diabetes, traumatic brain injury and depression.

6. The method of claim 1, wherein the stimulating agent is selected from the group consisting of: an anti CD3/CD28 antibody, Tetanus toxoid peptides or appropriately selected antigens, known to be contributors to the inflammatory process.

7. The method of claim 1, wherein the PBMC subpopulation identification comprises FACS analysis.

8. The method of claim 6 wherein, markers identified by FACS analysis are selected from the group consisting of: annexin V, B7-H1, B7-H3, B7-H4, B7-DC, B cells, memory B cells, CCR6, CD1c, CD4, CD8, CD11c, CD14, CD16, CD19, CD25, CD 27, CD28, CD30, CD39, CD40, CD56, CD62L, CD69, CD80, CD86, CD103, CD107, CD123, CD127, CD141, CD200, CTLA-4, CXCR3, Fas, FasL, FoxP3, GARP, GATA-3, GITR, GranzymeA, GranzymeB, HLA-DR, ICOS, IL-1β, IL-2, IL-4, IL-6, IL-10, IL-12, IL-12R, IL17, IL-17A, IL-21, IL-23, INFγ, KOS, memory CD4 cells, monocyte cells, naïve CD4 cells, NK cells, NKT cells, OX40, PD-1, PD-LI/II, Perforin, RORC, STAT-3 phosphorylation, STAT-5 phosphorylation, T-bet, TCR_{gd}, TGFβ, TNFα, T regulatory cells, T effector cells and any combination thereof.

9. The method of claim 1, wherein the PBMC subpopulation identification is done by functional assay.

10. The method of claim 9, wherein the functional assay is selected from the group consisting of: a proliferation assay, a suppression assay, confocal microscopy, Western blot, CTV staining, CFSE staining and PI assay.

11. The method of claim 10, wherein confocal microscopy determines the phosphorylation state of STAT-3 and STAT-5.

12. The method of claim 1, wherein the PBMC subpopulation identification is by PCR analysis.

13. The method of claim 1, wherein the profile includes PCR analysis of markers selected from the group consisting of: FoxP3, GATA-3, Granzyme, ICOS, IκBα, IKK, IL-2, IL-4, IL-6, IL-10, IL-12, IL-12R, IL-17A, IL-21, IL-23, INFγ, NFκb pathway, p50, PD-1, Perforin, RelA, RORC, T-bet, TGFβ, TNFα and any combination thereof.

14. The method of claim 1, wherein the subpopulation is selected from the group consisting of:
a) CD4⁺, CD127⁺ and T effector cells;
b) CD4⁺, CD127⁻ and T regulatory cells;
c) CD4⁺, CD127⁻, CD25^{low/neg} and nT regulatory cells;
d) CD4⁺, CD127⁻, CD25⁺⁺ and nT regulatory cells;
e) CD4⁺, CD127⁻, CD25⁺⁺, PD-1⁺ and tT regulatory cells;
CD14, CD19, CD11c and CD123;
g) CD56⁺, CD3⁻ and NK cells;
h) CD8⁺ and T cells;
i) CD4⁺, CXC3⁺, CCR6⁻, and Th1 cells;
j) CD4⁺, CXC3, CCR6⁺, CD161⁺ and Th17 cells;
k) CD4⁺, CXC3⁺, CCR6⁺, CD161⁺, Th1 cells and Th17 cells;
l) CD8⁻, CD4⁺ and T cells;
m) CD8⁺, CD4⁻ and T cells;
n) CD19⁺ and B cells;
o) CD14⁺ and monocytes;
p) CD4⁺, CD25^{high} and T effector cells;
q) CD4⁺, CD25⁻ and T effector cells; and
r) CD4⁻, CD8⁻, CD 19⁺ and B cells.

15. A method of monitoring the course of an immune mediated disease therapeutic agente therapy comprising:
a) isolating PBMCs from a subject previously or currently being treated for an immune mediated disease;
b) stimulating the PBMCs;
c) identifying subpopulations of PBMCs;
d) comparing data for step (c) to a subject response to the therapeutic agent; and
e) selecting a marker profile related to a positive response to the therapeutic agent, thereby monitoring the course of therapy in a subject.

16. Use of a Immunomics platform to predict response to an immune mediated disease therapeutic agent comprising:
a) isolating PBMCs from a subject treated for an immune mediated disease;
b) stimulating the PBMCs;
c) identifying subpopulations of PBMCs;
d) comparing data for step (c) to a subject response to a therapeutic agent; and
e) selecting a marker profile related to a positive response to therapeutic agent.

17. The method of claims 15 and 16, wherein the PBMCs are isolated from subjects treated with a biologic therapeutic.

18. A method of predicting response to an immune mediated disease therapeutic agent in a subject comprising:
a) obtaining a blood sample from a subject in need of therapy; and
b) analyzing the blood sample for a marker profile identified by the method of claim 1; wherein when markers associated with a positive response to the therapeutic agent are identified, a subject responsive to therapy is identified.

19. The method of claim 32, wherein the blood sample is analyzed using a method selected from the group consisting of:
a) a protein chip;
b) a gene expression chip;
c) protein expression;
d) detection of phosphorylation;
e) phosphorescence or luminesence;
f) immunobeads; or
g) a combination of all the above.

20. A protein or gene profile containing the markers identified in e) of claim 1.

21. An array containing one or more protein or gene markers of claim 20.

22. The array of claim 21 wherein the array is on a gene or protein chip.

23. A method for diagnosing an immune mediated disease comprising:
a) isolating proliferating blood mononuclear cells (PBMCs) from a subject diagnosed with an immune mediated disease;
b) stimulating the PBMCs;
c) identifying subpopulations of PBMCs;
d) comparing data from step (c) to subject diagnosis; and
e) selecting a marker profile related to a positive diagnosis of an immune mediated disease.
